(19) United States
(12) Patent Application Publication
(10) Pub. No.: US 2006/0084935 A1
(43) Pub. Date: Apr. 20, 2006

(54) Absorbing center for disposable diapers, process and machine to make them

(76) Inventors: Celso Junqueira Franco, Sao Paulo (BR); Ricardo Junqueira Franco, Sao Paulo (BR)

Correspondence Address: WILLIAM COLLARD;COLLARD & ROE, P.C., 1077 NORTHERN BOULEVARD, ROSLYN, NY, 11576, US

(21) Appl. No.: 10/529,926

(22) PCT Filed: Jul. 31, 2003
(86) PCT No.: PCT/BR03/00106

§ 371 Date: Mar. 31, 2005

(51) Int. Cl. A61F 13/15

(52) U.S. Cl. 604/368

(57) ABSTRACT

Improvement in absorbing center for disposable diapers, process and machine for its manufacturing, of the type made by cellulose flakes (11) adequately compacted forming a coverlet that, in its turn, presents an upper and a lower part with one or more capping (4-6) or still, with no capping, but, independently from such aspects, such center presents a plurality of absorbing gel grains (16) concentrated in distinct points (30) strategically distributed throughout the flake coverlet (11), where each point keeps a certain distance from the adjacent points, just as each point concentrates a certain number of grains, such number disposed in the medium part of the thickness (E) of the cellulose flake layer (11), where each absorbing gel point (16) is compacted at least on the upper side of the center (2), forming a cavity (31), being yet another characteristic of the present improvement the fact that such capping (4), upper and lower, presents the internal surfaces with a thing layer of absorbing gel grains (16), which are previously applied onto such internal surfaces using a sticking component or any glue, so that they may remain against the upper and lower surfaces of the flake coverlet (11).

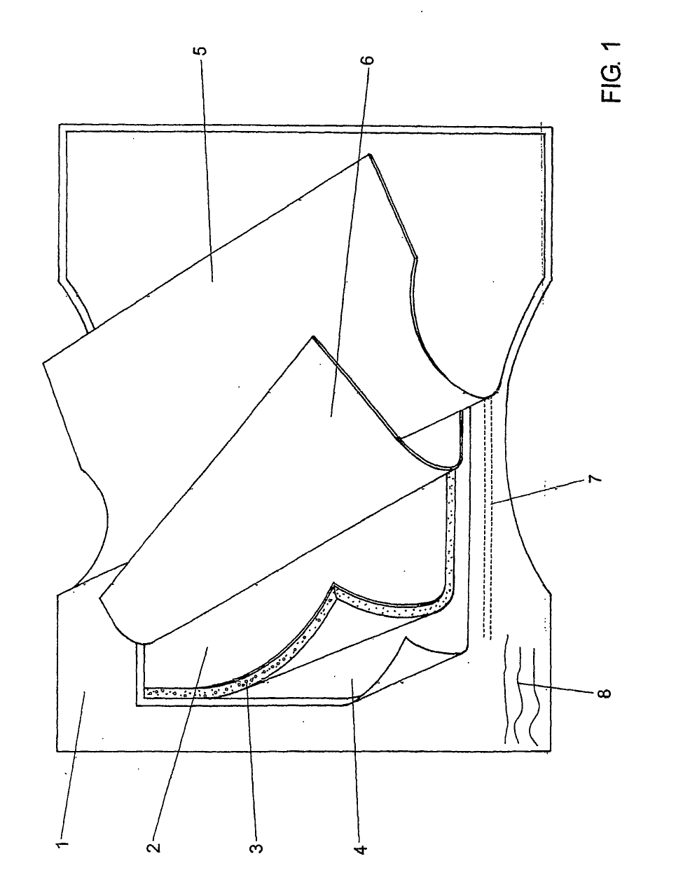

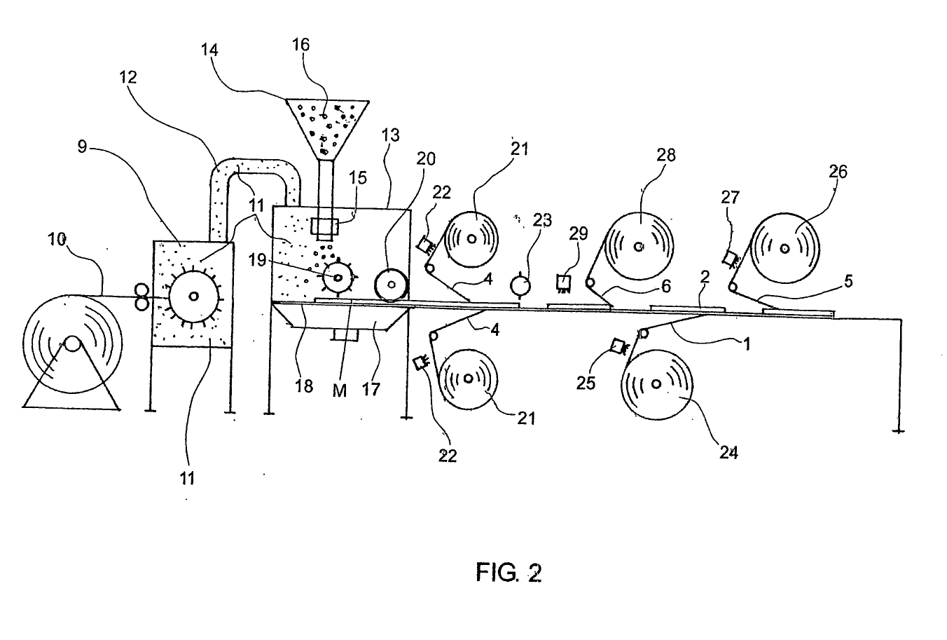

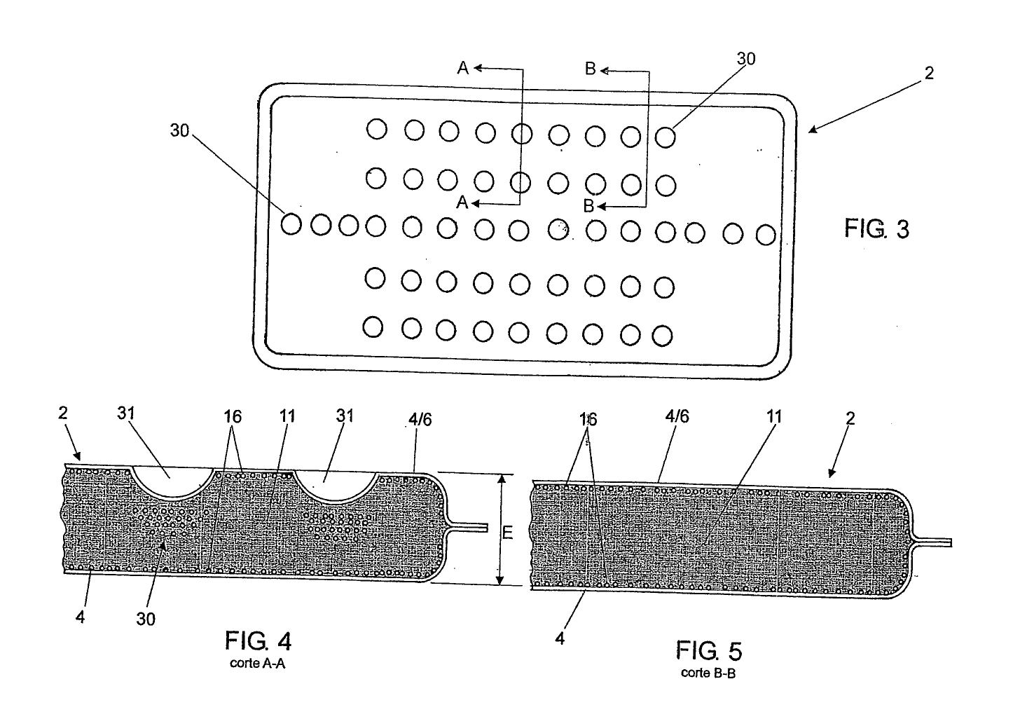

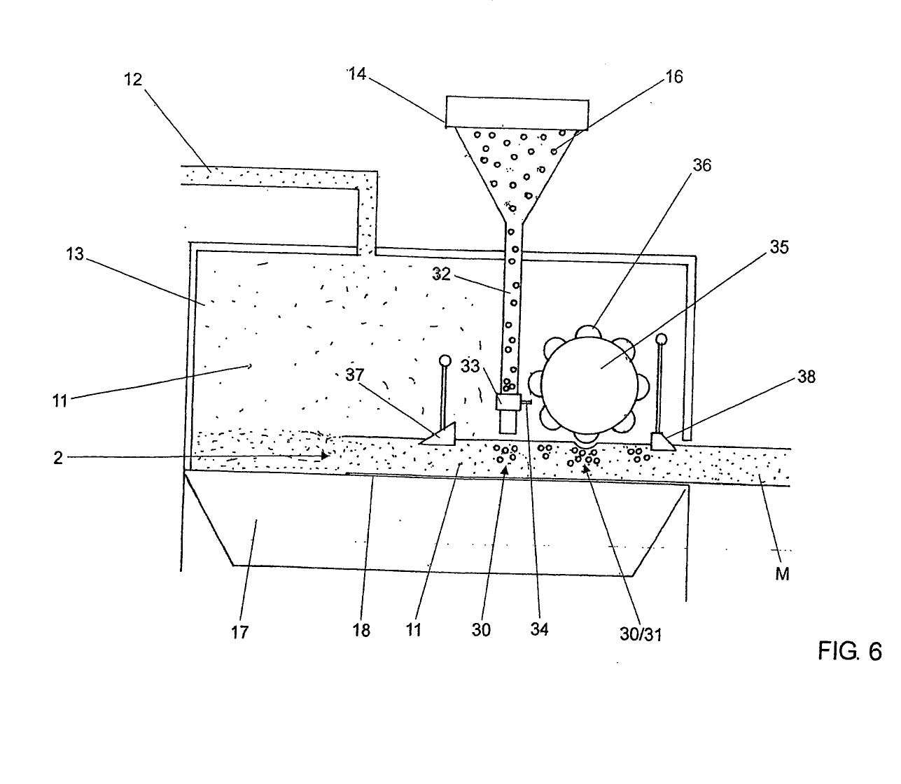

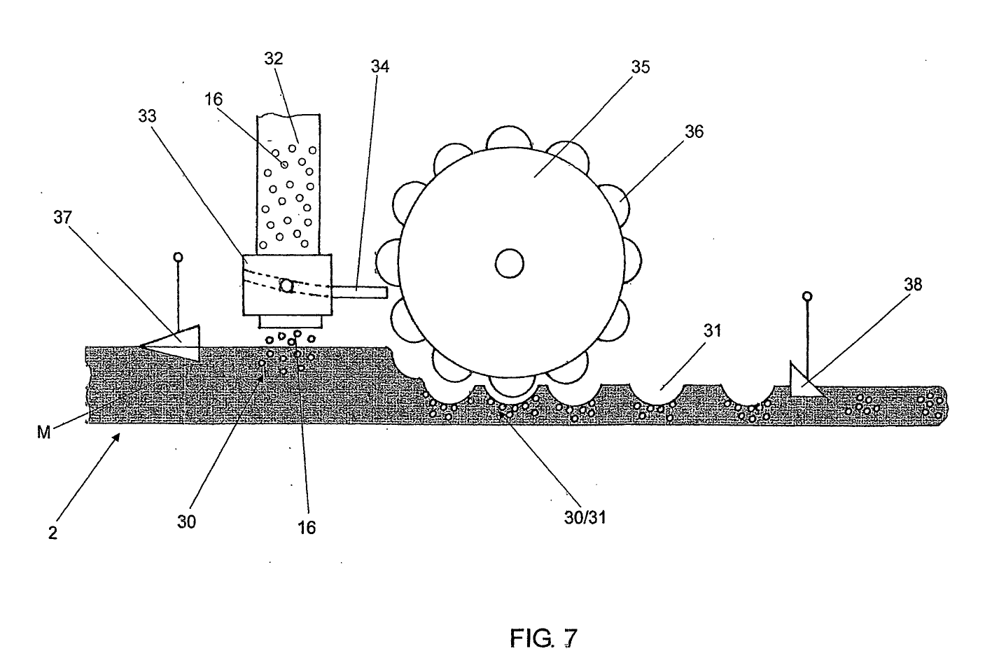

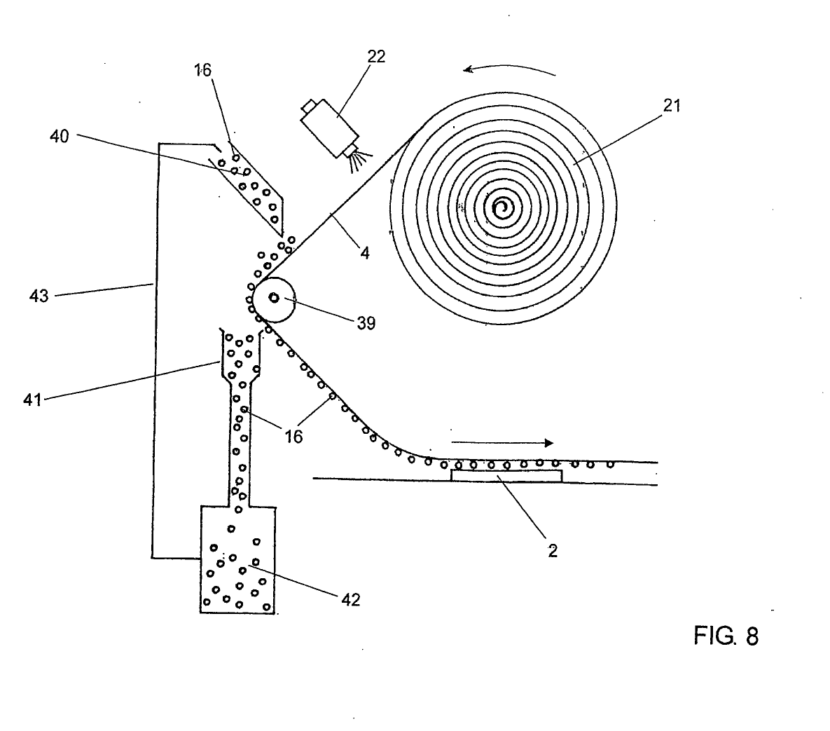

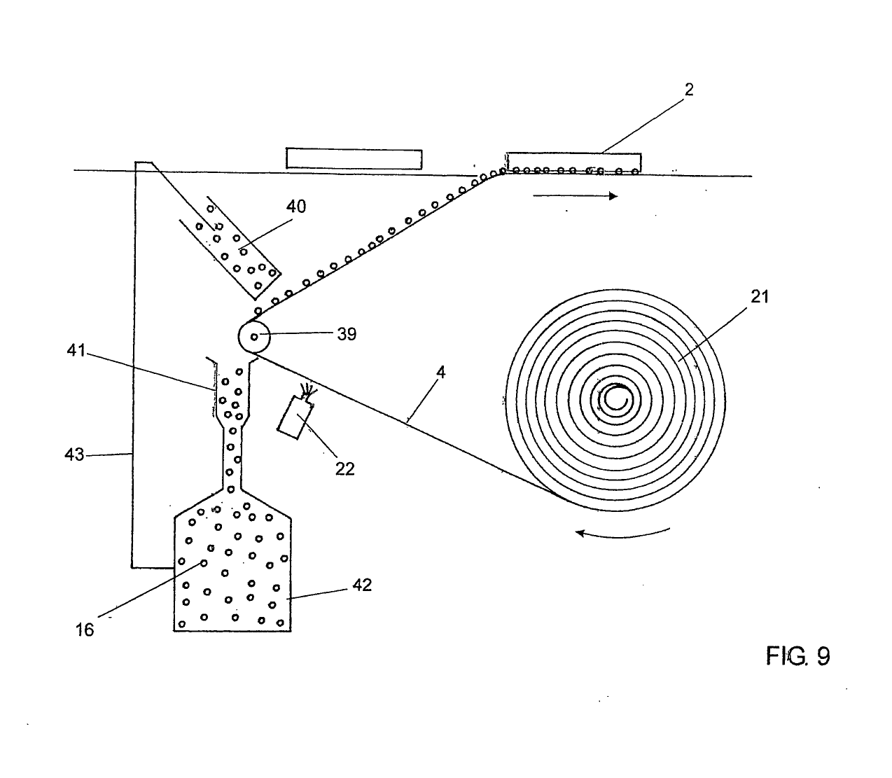

### FIELD OF THE INVENTION

Particularly, the present Invention refers to technical and functional improvements specially devised for the characterization of significant changes in the absorbing center of several disposable diapers, whether for children or adults, where the invention consists basically in finding means for the optimization concerning the allocation of the so called absorbing gel grains in more appropriate places and in a better way, with better conformation and confining them so as to increase their performance in the absorption of liquids, providing less contact between the humidity and the body of the user, avoiding this gel, whose texture is rather grainy, sandy-like and extremely itchy, from escaping, and therefore avoiding the leakage of those liquids, and reducing significantly the amount of absorbing gel grains applied, besides other complementary advantages.

### SITUATION OF THE TECHNIQUE

Presently there are different disposable diapers for children and adults, but all of them have one predominant part which is really pretty similar called the absorbing center composed of cellulose pulp fibers, which receives a chemical compost or polymer generically called absorbing gel, which are grains of a chemical product of silicon or other basis, with the ability to absorb many times its size or granulation, being even able to receive other components in this center.

The conventional centers, as it was mentioned before, are conformed based on cellulose pulp fibers and, therefore, such material, in its industrialization process, is de-fibered to present an appearance of flakes so that it can be mechanically mixed with the absorbing gel grains. This mixture, though homogenous, is made at random in disproportional quantities of each component.

There is no doubt that the center obtained in accordance with the situation of the present technique defines the necessary means for the ensemble to turn into an efficient unit when it comes to absorbing liquids; however, some inconveniences can be found during its use, the most important of which is the fact that there is no interaction of the encapsulation (confining) among the grains of absorbing gel and the fibers or cellulose pulp flakes, for such components are only mixed, and, consequently, there is not the “claw” effect between the two components, creating other inconveniences with that not only in the industrialization process, but also in the final usage of the center in the diaper, i.e., the diaper assembling process requires the use of machines with too many rotational and movable parts, where vibrations are highly common and, consequently, maintaining the grains stable making it difficult to control them, which, at the end of the process, results in the presence of such grains in unwanted places, not only in the diaper itself, but also in the working area and in the machine.

On the other hand, the most significant disadvantage takes place with the ready product, for some grains usually do not stay imprisoned in the center and, in such condition, may get in touch with the skin of the user causing itching and discomfort.

### OBJECTIVES OF THE INVENTION

The main objective of the present invention is to define a strategic way to place and distribute the absorbing gel grains in the coverlet formed by the flakes and cellulose pulp fibers.

It is also an objective of the invention that such distribution be made in smaller quantities, but in better positions and in a different way from the one that is presently adopted.

The result of this proceeding is an absorbing center with better and faster absorbing ability, once the absorbing gel grains are positioned only in the places where they are mostly needed.

This way, the user, child or adult, will have less contact with the humidity of the urine and, therefore, will suffer less unpleasant effects or damages to his/her health.

With the current improvement, another important advantage is achieved because the strategic placement of the grains results in the optimization of their stability and, along the way, the absorbing center obtained allows the grains to be maintained attached or practically encapsulated by the cellulose pulp fiber flakes. This way, in the processing phase as well as in the ready product, the absorbing center avoids or reduces the possibility of such gel grains escaping, providing superior efficiency compared to conventional centers.

### DESCRIPTION OF THE DRAWINGS

For a better understanding of the present Invention, a detailed description is made as follows, making reference to the attached drawings, where:

PICTURE 1 represents a view in schematic perspective pointing out the various layers present in a conventional diaper, showed only for figurative purposes;

PICTURE 2 shows a schematic view of a machine used for the manufacturing of diapers in general;

PICTURES 3, 4 and 5 respectively, portray an upper view and two details in amplified sections of an improved absorbing center according to the present invention; and

PICTURES 6 to 9 show several amplified views of segments of the machine in focus and that are directly related with the improvement at hand.

### DETAILED DESCRIPTION OF THE INVENTION

According to these illustrations and in its details, particularly picture 1, the present improvement applies to different diapers used by children and adults, such as that generically illustrated in picture 1, where it can be seen that it is composed of several basic parts, starting with a layer or external plastic film made of polyethylene (1) or other types of impermeable, isolated, or composed subs tracts, whose outline is tailored according to a specific shape, just as over its surface, an absorbing center (2) is stretched having been clearly obtained from a compost of cellulose pulp fiber, forming a kind of rectangular coverlet, and also receiving another component obtained from a chemical compost or polymer, traditionally called absorbing gel (3), which is basically grains of a chemical product of a silicon or other basis, with the ability to absorb many times its dimension or granulation, such as, for instance, fifty times its volume, still being able to receive in such center some other components.

As it was already said, the mixture of cellulose pulp fibers and absorbing gel form an absorbing center (2) that, in its turn, is still normally covered, in its lower part, by layers of thin paper such as tracing paper (4), also made of agglutinated cellulose, called “tissue”-type paper, which are is similar or resembling the traditional toilet paper.

In the external lower part of the diapers, which get in contact with the human body, there is a component called “tissue non-tissue” (TNT) or “non-woven” (5), which is a weave of threads of plastic chemical products, such as polypropylene or other materials, impermeable and with perforations which allow or promote the infiltration and passage of the liquids expelled by the users, for a further absorption in the lower layers. It is fundamental that this first layer of weave (TNT) (5) remains the driest the possible, under the best possibilities, so that it won't disturb or cause harm to the user, just as under this external layer (5) the diapers may receive a new film made of the same material “non-tissue” (TNT) or “non-woven” (6), called transferring layer or simply “transfer-layer”. This layer is meant to separate and improve the isolation between the lower external part of the diaper, which should be drier, and the internal part of it, where there is a concentration of the liquids, and which stay damp or wet when used. This isolation function prevents the urine from getting in touch with the body causing itching, skin rashes or other unpleasant consequences.

The diapers can also come with special components to avoid a migration of feces or urine from the internal parts of the diaper to the outside and are called fecal barrier or “leg cuff”. These barriers are normally constituted of “non-tissue” or “non-woven” compost already mentioned with elastic bands for a better adjustment to the human body and its union or attachment to the body or structure of the diaper and to avoid the harms caused by occasional leakages.

The body or structure of the diaper also comes with longitudinal elastic bands (7) to promote a better adjustment or better molding along the human body in two of its internal sides, but that are irrelevant for the present improvement. Just as irrelevant, most diapers have a sticker or sticking tape attached to their frontal part, many times colored and decorated (usually called frontal stripes or frontal “tape”) and still two lateral stickers or sticking tapes to fasten the parts to each other so that they stay fastened, stuck, or attached to each other and firmly placed along the body.

The forming elements of the diapers mentioned above, such as the plastics, the non-tissues; the transferring layers; the absorbing construction papers; the fecal barriers and other components, are normally and usually attached to each other by stickers or glue (8). Normally stickers called hot-glue or “hot-melt” are used for this purpose. These stickers are polymers or composts that are solid under normal temperature, but that melt once heated, changing its viscosity and physical-chemical aspects and then used in this state. Among its main characteristics, we can point out the facility to be applied, the immediate or instantaneous result of the gluing, and the strong attachment between the parts. Most of the times, the hot glue that is used has the permanent attachment property, i.e., it remains continuously adhered to touch and independently from other factors, mainly moist. This glue is also called “permanent sticking”, permanent attachment, self-sticking, “pressure-sensitive”, among other names.

The automated production of diapers in high speed requires specific equipment, such as that illustrated in picture 2, already largely spread worldwide and generally having basic parts, starting from a mill or de-fiberer (9), which can be made of a disentangling piece or hammer or other methods, which transforms the cellulose pulp (10), prepared in rolls or leaves, particularly in small texture or fibers, herein called flakes (11).

The cellulose pulp is normally supplied in bobbins of variable width and other proportions, long or short fibers with different levels of humidity, besides other irrelevant factors.

The de-fiberer (9) is connected though a feeding duct system (12) to another set called agglomerating or agglutinating piece (13), which, in its turn, is also connected to an upper tray (14) and its respective regulator (15) so that in the internal part of the agglomerating piece (13) a continuous flux of absorbing gel grains (16) may be introduced along with the flakes (11) and, in the internal part of the so called agglutinating piece (13) such components are mixed in a disperse way and, at the same time, the agglomeration of the cellulose pulp already de-fibered or in flakes (11) also takes place.

The de-fiberer (9) generally consists of a closed cabinet sometimes with transparent covers (so that one can observe the performance of this cellulose pulp), with a lower vacuum chamber (17) mounted over a screen (18) usually made of plastic and placed as a conveyor belt and where the fibers gather or agglomerate and are transported to the following phases. Sometimes this chamber has a rotating device (19) with metal edge or edges to make room in the pulps that are formed for a better penetration of the absorbing gel (16) and concentration of the pulp. Almost at the exit of this chamber and over the conveyor belt (18) there is a compressor (roll) (20) of proportional dimensions to its normality and use, normally made of metal, which is supposed to partially compact the cellulose fibers and start the conformation process, practically transforming it in the absorbing center (2) for its final use as an absorbing coverlet and also to allow its passage through the exit opening of such chamber.

Depending on the equipment, it is possible to use two or more de-fibering mills (9) to produce and allocate the cellulose pulp coverlets in layers and trying, by doing so, to let the absorbing gel lay among these layers.

Among other models of coverlet formers or absorbing coverlet agglutinating piece for the production of diapers, there is also rotating former (which is complemented or is a variation of the same process), with several molds imprinted on it, with the desired coverlet shapes and on it allocated throughout its length also with a vacuum system with screen to suck retain, transport and dispense the cellulose pulp, shaping it in the desired, defined and specified conformations in each of these molds allocated in this rotating former, but that is not configured under the inventive circumstances of the present improvement, for it takes place after the distributing operation of the absorbing gel grains in the forming of the coverlet.

The tray (14) for stocking up and gel grains feeding to the system may radically vary in its dimensions and in other constructive details, mainly on what refers to the regulator (15) which can be one of mugs, openings over rotational metals, strangling of the piping, several valves, among others. Normally and mostly, the gel grains dosed are completely dispersed over the cellulose fibers, mixing up almost homogenously, though uniform with them.

Therefore, after its dosing through several methods previously mentioned, the gel is thrown over the fibers of the pulp by gravity force. It may be scattered or completely disseminated over the totality of the body of the pulp being formed or, occasionally, concentrated over a more centralized area the conformation of this pulp and that is established also more centralized in a longitudinal perspective during the assembling and in the diaper already accomplished or finished. This way, when the cellulose pulp and the gel grains go through the compressor roll (20), they get together and remain mixed in a disperse way, generalized and relative or partially homogenized, or even, occasionally, as it was said before, a little more concentrated in the central area of the formed pulp, longitudinally. The absorbing gel (16) usually stays, this way, mixed to the cellulose pulp (11) almost totally, in length, width as well as in thickness, without a specific control system and without the possibility of measuring or defining the exact spots where one wants to place them for a bigger and better absorption and distance from the user's body.

The machine depicted in picture 2 includes means for the allocation of the “tissue” papers (4), which can be called ‘de-bobbinators’ (21). These papers can only be placed over the pulp and may even come with both conformations (under and over the cellulose pulp with gel). The purpose of these papers is to promote, during the production, larger facility in the transportation of the pulp with gel in the assembling process, keeping the pulp is with gel compact, avoiding the escape of cellulose pulp fibers and gel during their transportation. After the fabrication and when in use, it has the purpose of improving the look of the diaper, preventing its agglutinated cellulose fibers from parting or breaking and forming a huge agglomeration or shapeless ball under the legs of the user when receiving liquid and, still, providing a more pleasant use of it. It also serves to facilitate the distribution of the liquids and promoting more absorption. Operationally, these de-bobbinators (21) are placed over or under the main machine or in both places and where the “tissue” paper roles are located. They may receive liquid glue (22) (also called cold glue) with acrylic or PVA basis and even water or hot glue sprinkled or flattened in the shape of sticking blades. This glue or sticker promotes the attachment of the “tissue” paper (4) to the cellulose pulp coverlet, promoting more steadiness or compactness of the set. The necessary strength for the be-bobbing of these rolls of paper (21), as well as its feeding in the process, is made by the main machine itself, which tracks and moves the entire set. The set is made of cellulose pulp with gel, with the addition of “tissue” paper or papers (4) occasionally sticking, is cut by a cutting knife (23), normally rotational, in the specified and determined shapes and sizes, after which the absorbing center (2) is definitely formed.

The machine still includes another de-bobbinator (24) of plastic film (1) of polyethylene already mentioned. This plastic film constitutes the external part of the diapers. The roll with the plastic film (1), which is inserted through the lower part, i.e., the de-bobbinator (24) is usually placed under the main machine. As the plastic film moves, it also receives hot glue (25) through sprinkling, blades, or continuous traces in the places and necessary and determined amounts. To this plastic (1), the absorbing cellulose center (2) is attached, made of cellulose pulp (11) with gel (16) and, occasionally, with the “tissue” paper (4).

On the upper part of the machine, there is another de-bobbinator (26) for the “tissue non-tissue” or “non-woven” (5) film. The same way as the plastic films, the “tissue non-tissue” or “non-woven” may be impregnated with hot glue (27), in the desired places and quantities, through sprinkling, through lines, or through blades, at the time of its de-bobbing and inclusion in the forming of the diapers. As it was previously mentioned, this material will be in direct contact with the human body.

As it was previously said, the diaper may have a layer called “transfer layer” or transferring layer (6) and, for that matter, the main equipment includes a de-bobbinator (28) for that layer, including the gluing phase (29), being this layer also a tissue non-tissue and its positioning is made prior to the external tissue non-tissue layer (5) and placed under it. This film, as it is de-bobbinated may receive the hot glue before it is inserted in the body or structure of the diaper. This film usually has its width smaller or the same size as the width of the cellulose pulp coverlet (2). Its moving is also made by the traction or power exerted by the main machine.

The allocation of the other components for the production of the fecal barrier, the plastic tape and frontal sticker (or “frontal tape”), the side sticking tapes, the elastic bands and other occasional components has no relevance to the details of the present improvement and, therefore, are not herein specified.

According to pictures 3, 4 and 5, the present improvement us applied on a center (2) formed by cellulose flakes (11) adequately compacted forming a coverlet that, in its turn, presents the upper and lower parts with one more capping (4-6) or still, with no capping at all, though characterized for the fact that the coverlet initially presents a plurality of absorbing gel grains (16) concentrated on distinct points (30) and strategically distributed throughout the so called flake coverlet (11), where each points keeps a certain distance prom the adjacent points, just as each point concentrates a certain number of grains, such quantity being disposed in the medium part of the thickness (E) of the flake layer (11), where each absorbing gel point (16) is compacted at least on the upper side of the center (2), forming a cavity (31).

Another characteristic of this improvement, as shown in pictures 4 and 5, is the fact that the capping (4), upper and lower, present their internal surfaces with a thin layer of absorbing gel grains (16), which are previously applied onto such internal surfaces using an adhering component or any glue, in a way for them to be both on the upper and lower surfaces of the flake coverlet (11).

Therefore, what is being initially claimed is a center (2) with a better distribution and allocation of the absorbing gel grains over the cellulose de-fibered pulp or flakes so as to have larger and better results in the absorption of liquids and maintenance of the “dryness” in the diapers, besides other advantages.

To obtain the improved center described above, it is necessary for the machine exemplified in picture 2 to be complemented with some specific mechanical devices so that the complementary phases can be accomplished and, to do so, pictures 6 and 7 show some mechanical complements specially introduced for the distribution of the absorbing gel (16) to be made over the center (2) which will become the core of the diaper, i.e., the agglomerating unit (13) is modified so that its tray (14) can present its lower part equipped with multiple exits represented by a chain of vertical dispenser spouts (32) strategically positioned in a transversal way, each having a lower valve or regulating device (33) and respective trigger (34) turned back and facing a compressor roll (35) placed at the end of the cellulose flake coverlet forming chamber (11) and before or after their exit from the agglomerating piece (13) or still, out of it, just as the external diameter of the compressor roll (35) distributes the plurality of compacting protuberances (36), which occupy cooperating positions so that, initially, they may start the triggers (34) of the regulators (33) so that the predetermined doses of gel grains (16) may be dispensed over the flake coverlet (11), forming the concentrated points (30) that, in turn, logically on in tune, each point (30) is compacted (31) by the same protuberances (36), in a way that, before the dispenser spouts (32) and after the compressor roll (36), layers with the same number of route tracers (37) and route closers (38) are displayed, being those placed before the unloading of the gel grains are cooperating to form furrows or tracks that flow under the regulators (32) and, with that, the upper cellulose pulp layer is opened to guarantee and make sure that the absorbing gel grains (16) be placed in the interior part of the flake coverlet, while the second set of route closers (38) do exactly the opposite, i.e., close the so called track, covering each compaction (31) so that the absorbing gel grains (16) may be encapsulated.

One can make the specified drawing or design of the gel points (30) in the body or structure of the center (2) of the diaper as desired, through the results that are meant to be obtained such as, for example, illustrative and hypothetic, picture 3 shows that in the first 20% part of the longitudinal length of the diaper coverlet, and that when using it placing on the belly and near the navel of the user where the urine penetration is almost non-existent, a small number of central gel points may be used, starting from the beginning of the diaper coverlet and, after that, this number may gradually or drastically increase in the rest of the length of the center (2), but these and other details do not alter the present improvement.

The same situation takes place with the back part of the center (2), i.e., in the last 20% of its length in the back, for there as well the presence of humidity is very reduced. Note that the specifications concerning the determination of the amount of absorbing gel and the places where it will be placed may present a number of configurations. The central part of the absorbing center is where the flux of liquids is concentrated so it is also where there should be a higher incidence of the absorbing gel (16). This way and also hypothetically and for illustrative and figurative effects, it could be specified that for an occasional width of absorbing coverlet of 90 mm, four gel points, with a distance of 20 mm between each of them and leaving 5 mm in each of the lateral borders without gel. These gel-applied points could be repeated, also at a distance of 20 mm between each of them and along the total 60% of the absorbing coverlet (except from the 20% of each of the edges of the absorbing coverlet—from its beginning and from its end already mentioned).

Hypothetically, for clarification purposes, in an illustrative or figurative sense, it could be exemplified that each of these absorbing gel points (30) could be 5 mm wide and weigh approximately 0.0375 gr. each.

A cause and also a consequence of placing protuberances (36) over the compressor roll is that they will produce the deepening (31) or the compression of the gel grains (16) inside the pulp (11). This procedure is fundamental, for the gel will penetrate in the pulp, remaining well attached there and facilitating the absorption of the liquids. Another important reason for this compression is that the gel will remain relatively pressed inside the pulp (11), preventing it from falling or coming out of the pulp, which is where it should be effectively fixed. By doing so, unnecessary waste is avoided, such as its falling over the machine or on the ground, generating unpleasant dirt; damaging the sharpening of the cutting knives ahead (and prolonging its running time and avoiding the need to stop for sharpening) or still, falling inside shafts or other mechanisms; falling over the glue and damage the gluing process and causing leakage of the liquids placed there, and still and mainly not performing its real function for which it was placed there, which is to absorb liquids. When the absorbing gel comes out of the pulp, reality is faked, i.e., it is as if a certain specified amount of gel having been placed to produce certain absorption result, when, in fact, a smaller amount has been applied.

As it was already mentioned, the center of the diaper may have a leaf of “tissue” paper (4) over the cellulose paper (4) to strengthen it and provide a more resistant and absorbing structure. It may also have, simultaneously or isolated, a transferring layer of tissue non-tissue (6) (“transfer layer”) for liquids and which is the connection of the external part of the diaper which gets in touch with the human body with its inner part or hub and where liquids should be concentrated or retained. Many companies do not use the tissue non-tissue materials for the transfer layer nor even the “tissue” paper (4), due to the high prices of those, lack of technology marketing or technological choice, besides other implications.

Nowadays, these materials, the “tissue” paper (4) as well as the tissue non-tissue (6) only remain attached to the lower layers, i.e., the absorbing coverlet, by simple overlapping or through use of glue or even water.

Considering that it is of fundamental importance that the liquids dispensed be absorbed as quickly as possible by the previously mentioned absorbing materials in the diapers, not only to prevent them from leaking out of them, but also and mainly to avoid or reduce as much as possible their contact with the human body, the present improvement includes other betterments, obviously to increase even more the efficiency at absorbing liquids in the diapers, being such betterments shown in details in picture 8, where the application of absorbing gel grains (16) over the hot glue recently applied (22) on the tissue non-tissue or “tissue” paper (4) transferring layer can be noticed. This applied gel adheres to the glue on the pre-selected and determined desired places and quantities, and that will absorb the liquids better and faster. This place is normally located in the central longitudinal areas of the diaper, with small differences in spot, due to sexual variation of the users. Notice that the absorbing gel (16) remains with its grains exclusively facing the interior part of the diaper and near the areas of more intense absorption. This exclusive method promotes a faster initial absorption of the liquids disposed there. This improvement can be defined as first and main absorption.

As a consequent or complementary function of this method, the “tissue” paper (4), of lower cost and rather frail consistency, with the reception of the hot-melt “permanent-sticking” sticker through sprinkling, lines or blades, may behave in a much stronger and resistant way, improving its performance in the safety and final quality of the absorption and resistance of the diaper. If the tissue non-tissue is used, the diaper may also be stronger, thicker, and will show a better performance in its function of embodying and absorbing liquids quickly. In a preferred construction, it is necessary for the application of the absorbing gel grains (16) over the corresponding layer (4) surface that a guide-roll (39) be used placed between the paper roll (21) and the horizontal part of the machine so that the layer (4) may form a “V” angle and, in the anterior area of such guide-roll, a glue applier (22) is placed while after the guide-roll an absorbing gel grains (16) unloading mouthpiece (40), whose excess thrown over the hot glue is collected a little bit under that by another similar mouthpiece (41) with a lower recipient (42) connected or not to an aspiration system, so that the grains may return (43) from the last one to be used again by the mouthpiece (40).

As a further complement of the purpose of better performance of the diapers, in the sense of larger, better and faster absorption, the improvement plans, yet as a complement, as illustrated in picture 9, the placing of gel in the deeper layers and in the inside of the diaper coverlets, i.e., on the “tissue” paper (4) next and anterior to the external construction and protection plastic (1). It composes the lower layer of the gel and cellulose pulp absorbing coverlet, on the one side, and the external plastic on the other, as described before.

The same way as previously mentioned, also using some complementary equipment like the one in picture 8, absorbing gel grains (16) shall be applied and glued over the internal and lower “tissue” paper (4) (pic. 9), which gets in touch, on one side, with the cellulose pulp (11) and, on the other side, with the external plastic (1) of the diaper, a portion of “permanent-sticking” hot glue (22) on the desired place and amount, by means of sprinkling, blades, lines, or other means. On the immediate sequence, absorbing gel grains (16) are applied over this glue (22), remaining attached to its upper surface the grains with which it may get in touch. The excess shall be removed by a receptacle (41) with suction system or not and returned to the lower gel tank (42) and later transported (43) to the mouthpiece (40) for a new use.

The process of making the absorbing center described above is made of the following steps:

a) preparation of the cellulose pulp (10) in rolls or leaves, preferably with the preparation in the shape of bobbin, where the cellulose pulp presents longer or shorter fibers, with different levels of humidity, besides other irrelevant factors;

b) automatic feeding of the cellulose pulp (10) in a mill or de-fiberer (9), which can be made of a disentangling piece or hammer or other methods, which turn the cellulose pulp (10) into small texture or fibers, herein, called flakes (11);

c) introduction of the flakes (11) and absorbing gel grains (16) in the interior of an agglomerating or agglutinating piece (13);

e) transformation of the flakes (11) in a continuous coverlet (M) on the inside of the agglomerating piece (13) and, for that purpose, such flakes (11) are aspired by a vacuum system (17) against a conveyor belt (18) over which such continuous coverlet is formed (M) which, in its turn, is softly compacted and moves continuously forward;

f) allocation of the capping or “tissue” paper (4) through the use of de-bobinnators (21) and glue (22), so that these papers can be placed only over the pulp, or be placed under the pulp, or may still have both simultaneous conformation over and under the cellulose pulp with gel;

g) the coverlet (M) made of cellulose pulp with gel, added with “tissue” paper or papers (4), occasionally sticky, is cut or segmented by a cutting knife (23), usually rotational, in the determined and specified shapes, after which the absorbing center (2) is effectively formed;

h) disposing the centers (2) over a plastic film (1) made of polyethylene, which constitutes the external part of the diapers, just as such plastic film (1) is inserted from below, i.e., the de-bobbinator (24) is usually positioned under the main machine, where the plastic film (1) moves and receives the hot glue (25), by means of sprinkling, blades or continuous traces, in the determined and necessary places and amounts and, therefore, to such plastic film (1) the absorbing cellulose center (2) is attached made of cellulose pulp (11) with gel (16) and occasionally “tissue” paper (4);

i) application of a tissue non-tissue or non-woven upper layer (5) through a de-bobbinator (26), using hot glue (27) as well, on the desired places and quantities, by means of sprinkling, lines or blades, when making its de-bobbing and insertion in the assembling of the diapers;

j) as an option, a layer called “transfer layer” or transferring layer (6) is included, which is applied through a de-bobbinator (28) and its respective gluing (29), being this layer also a tissue non-tissue and its positioning being made anterior to the external tissue non-tissue layer (5) and placed under it;

k) allocation of the other components for the making of the diaper: fecal-barrier, frontal sticking plastic tape (“frontal tape”) and the lateral sticking tapes, the elastic tapes and other occasional components that vary according to the kind of diaper.

The steps listed above are rather common in many processes used in the making of diapers and respective absorbing center, but the present process is characterized by the fact that it involves the following complementary steps between step (e) and step (f):

e1) creation of longitudinal furrows in the upper part of the coverlet (M), such furrows being placed in parallel among each other and starting before the tray (16), where they are made by route tracers (37);

e2) disposing a plurality of absorbing gel grains (16) concentrated in distinct points (30) and strategically distributed throughout each longitudinal furrow of such coverlet (M), where each point remains at a certain distance from the adjacent points, just as each point concentrates a certain number of grains, being such number disposed in the medium part of the thickness (E) of the coverlet (M);

e3) compacting of each point (30) of absorbing gel (16), such compacting being made at least on the upper side of the coverlet (M) forming a cavity (31):

e4) closing of the furrows with route closers (38) capping each compacting (31) so that the absorbing gel grains (16) may be encapsulated;

f1) application of absorbing gel grains (16) over the recently applied hot glue (22) on the upper transfer layer of the tissue non-tissue or on the “tissue” paper (4), such gel applied there attaching to the glue, only on the pre-selected and determined desired places and quantities, which shall better and faster absorb the liquids, just as this place is usually located on the central longitudinal areas of the diaper with small differences in place, due to sexual variation of the users, but, in any case, the absorbing gel (16) remains with its grains exclusively turned to the inside of the diaper and near the areas of larger absorption;

f2) putting gel on the deeper layers and on the inside of the coverlets of the diapers, i.e., on the “tissue” paper (4) on the lower and anterior side to the external construction and protection plastic (1).

Steps (e1) to (e4) may take place inside or outside the agglutinating piece (13).

As it can be noticed, after all that has been exposed and illustrated, the present improvement includes sensitive changes to the absorbing center (2) of the diaper, as well as equivalent changes in its equipment and manufacturing process, aiming with this at obtaining the following characteristics and advantages:

a) placing of the absorbing gel grains in smaller amounts inside the diapers, but in better positions and in a different way from the one that is presently done;

b) obtaining a better and faster absorbing diaper, with absorbing gel grains put only in the places where it is most necessary;

c) less contact between the user and the wet parts of the diaper, be it for children or for adults;

c) preventing the absorbing gel grains from escaping from the interior of the center of the diaper and, therefore, preventing such grains from getting in touch with the skin of the user;

d) avoiding leakage of liquids;

e) avoiding loss or uselessness of the absorbing gel grains, for they are located in parts of the diaper where the humidity doesn't reach or needs to reach;

f) reducing the need of maintenance, stops, and costs of the diaper making equipment, for it prevents the gel from damaging the cutting knives by wearing them out, or from getting to shafts and reducing their running time, from dirtying these or making them need more cleaning and other maintenance measures, as also from blocking or damaging the vacuum conveyor belt and pulp former.

1. Improvement in absorbing center for disposable diapers, such improvement can be used in any kind of absorbing center (2), normally made of cellulose flake (11) properly compacted, forming a coverlet that, in its turn, presents an upper and a lower part with one or more capping (4-6) or still, with no internal capping; wherein the formed coverlet initially presents a plurality of absorbing gel grains (16) concentrated on distinct points (30) and strategically distributed throughout such flake coverlet (11), where each point keeps a certain distance from the adjacent points, just as each point concentrates a certain number of grains, such number disposed in the medium part of the thickness (E) of the cellulose flake layer (11), where each absorbing gel point (16) is compacted at least on the upper side of the center (2), forming a cavity (31).

2. Improvement in absorbing center for disposable diapers, according to claim 1, wherein when the absorbing center (2) includes capping (4-6), presents the internal surfaces of such capping with a thing layer of absorbing gel grains (16), which are previously applied onto such internal surfaces using a sticking component or any glue, so that the grains (16) may remain against the upper and lower surfaces of the flake coverlet (11).

3. Improvement in absorbing center for disposable diapers, according to claim 1, wherein the capping (4) with absorbing gel grains (16) is supposed to be placed on the upper and lower part of the coverlet and between the flake coverlet (11) and the plastic film (1).

4. Improvement in absorbing center for disposable diapers, according to claim 1, wherein the capping (4) with absorbing gel grains (16) is supposed to be placed on the upper part and between the cellulose flake coverlet (11) and the tissue non-tissue or “non-woven” (5).

5. Process for the making of the absorbing center described in claim 1, composed of the following steps:
a) preparation of the cellulose pulp (10) in rolls or leaves, preferably with the preparation in the shape of bobbin, where the cellulose pulp presents longer or shorter fibers, with different levels of humidity, besides other irrelevant factors;
b) automatic feeding of the cellulose pulp (10) in a mill or defiberer (9), which can be made of a disentangling piece or hammer or other methods, which turn the cellulose pulp (10) into small texture or fibers, herein called flakes (11);
c) introduction of the flakes (11) and absorbing gel grains (16) in the interior of an agglomerating or agglutinating piece (13);
e) transformation of the flakes (11) in a continuous coverlet (M) on the inside of the agglomerating piece (13) and, for that purpose, such flakes (11) are aspired by a vacuum system (17) against a conveyor belt (18) over which such continuous coverlet is formed (M) which, in its turn, is softly compacted and moves continuously forward;
f) allocation of the capping or “tissue” paper (4) through the use of de-bobbinators (21) and glue (22), so that these papers can be placed only over the pulp, or be placed under the pulp, or may still have both simultaneous conformation over and under the cellulose pulp with gel;
g) the coverlet (M) made of cellulose pulp with gel, added with “tissue” paper or papers (4), occasionally sticky, is cut or segmented by a cutting knife (23), usually rotational, in the determined and specified shapes, after which the absorbing center (2) is effectively formed;
h) disposing the centers (2) over a plastic film (1) made of polyethylene, which constitutes the external part of the diapers, just as such plastic film (1) is inserted from below, i.e., the de-bobbinator (24) is usually positioned under the main machine, where the plastic film (1) moves and receives the hot glue (25), by means of sprinkling, blades or continuous traces, in the determined and necessary places and amounts and, therefore, to such plastic film (1) the absorbing cellulose center (2) is attached made of cellulose pulp (11) with gel (16) and occasionally “tissue” paper (4);
i) application of a tissue non-tissue or non-woven upper layer (5) through a de-bobbinator (26), using hot glue (27) as well, on the desired places and quantities, by means of sprinkling, lines or blades, when making its de-bobbing and insertion in the assembling of the diapers;
j) as an option, a layer called “transfer layer” or transferring layer (6) is included, which is applied through a de-bobbinator (28) and its respective gluing (29), being this layer also a tissue non-tissue and its positioning being made anterior to the external tissue non-tissue layer (5) and placed under it;
k) allocation of the other components for the making of the diaper: fecal-barrier, frontal sticking plastic tape (“frontal tape”) and the lateral sticking tapes, the elastic tapes and other occasional components that vary according to the kind of diaper.
wherein it involves the following complementary steps between step (e) and step (f):
e1) creation of longitudinal furrows in the upper part of the coverlet (M), such furrows being placed in parallel among each other and starting before the tray (16), where they are made by route tracers (37);
e2) disposing a plurality of absorbing gel grains (16) concentrated in distinct points (30) and strategically distributed throughout each longitudinal furrow of such coverlet (M), where each point remains at a certain distance from the adjacent points, just as each point concentrates a certain number of grains, being such number disposed in the medium part of the thickness (E) of the coverlet (M);
e3) compacting of each point (30) of absorbing gel (16), such compacting being made at least on the upper side of the coverlet (M) forming a cavity (31);
e4) closing of the furrows with route closers (38) capping each compacting (31) so that the absorbing gel grains (16) may be encapsulated;
f1) application of absorbing gel grains (16) over the recently applied hot glue (22) on the upper transfer layer of the tissue non-tissue or on the “tissue” paper (4), such gel applied there attaching to the glue, only on the pre-selected and determined desired places and quantities, which shall better and faster absorb the liquids, just as this place is usually located on the central longitudinal areas of the diaper with small differences in place, due to sexual variation of the users, but, in any case, the absorbing gel (16) remains with its grains exclusively turned to the inside of the diaper and near the areas of larger absorption;
f2) putting gel on the deeper layers and on the inside of the coverlets of the diapers, i.e., on the “tissue” paper (4) on the lower and anterior side to the external construction and protection plastic (1).

6. Process for the making of the absorbing center, according to claim 5, wherein steps (e1) through (e4) may take place inside or outside the agglutinating piece (13).

7. Machine for the making of the absorbing center described in claim 1 and to make the process described in claim 5, composed of a mill or a de-fiberer (9), which can be made of a disentangling piece or hammer or other methods, which turn the cellulose pulp (10), prepared in rolls or leaves, into small texture particles or fibers, herein, called flakes (11); just as the de-fiberer (9) is connected through a feeding duct system (12), to another set called agglomerating or agglutinating piece (13) which, in its turn, is also connected to an upper tray (14) with a lower vacuum chamber (17) which, along with a conveyor belt (18), form the coverlet (M), which slides to pass through other parts of the machine, which includes upper and lower de-bobbinators (21) with their respective glue disposers (22) for the placing of the “tissue” papers (4), upper and lower, after which such coverlet is cut by a cutting knife (23), usually rotational, in determined and specified shapes and sizes, after which the absorbing center (2) is effectively formed, which, in its turn, through other bobbins (24-26-28), receives other blade materials layers (1-5-6), using other glue disposers (25-27-29); wherein the tray (14) presents its lower part equipped with multiple exits represented by a chain of vertical dispenser spouts (32), strategically placed in a transversal way, each having a lower valve or regulating device (33) and respective trigger (34) turned back and facing a compressor roll (35) placed at the end of the cellulose flake coverlet forming chamber (11) and before or after their exit from the agglomerating piece (13) or still, out of it, just as the external diameter of the compressor roll (35) distributes the plurality of compacting protuberances (36), which occupy cooperating positions so that, initially, they may start the triggers (34) of the regulators (33) so that the predetermined doses of gel grains (16) may be dispensed over the flake coverlet (11), forming the concentrated points (30), that, in turn, logically on in tune, each point (30) is compacted (31) by the same protuberances (36), in a way that, before the dispenser spouts (32) and after the compressor roll (36), layers with the same number of route tracers (37) and route closers (38) are displayed, being those placed before the unloading of the gel grains are cooperating to form furrows or tracks that flow under the regulators (32) and, with that, the upper cellulose pulp layer is opened to guarantee and make sure that the absorbing gel grains (16) be placed in the interior part of the flake coverlet, while the second set of route closers (38) do exactly the opposite, i.e., close the so called track, covering each compaction (31) so that the absorbing gel grains (16) may be encapsulated.

8. Machine for the making of the absorbing center, according to claim 7, wherein the mouthpiece (41) is conjugated with as aspiring and transporting system by flow of air combined with the storage (42) and with an absorbing gel grains (16) returning conductor (43) to the mouthpiece (40).
